(19) United States
(12) Patent Application Publication
(10) Pub. No.: US 2012/0179226 A1
(43) Pub. Date: Jul. 12, 2012

(54) Therapeutic and Safety Grip Device

(76) Inventors: R. William Graham, Oklahoma City, OK (US); Jane C. Graham, Oklahoma City, OK (US)

(21) Appl. No.: 12/985,382

(22) Filed: Jan. 6, 2011

(51) Int. Cl. A63B 23/16; A61F 7/00

(52) U.S. Cl. 607/77; 607/111

(57) ABSTRACT

A grip device for use in one's hand includes a compressible member of a size to fit within one's hand and capable of being squeezed by the hand and a thermal element operably connected to the compressible member which is equipped for generating a non-ambient temperature for a sustained period during such period said device remains compressible. A sensor and transmitter circuitry is connected to the compressible member which when the grip device is compressed sends a signal indicative that compression is taking place. A computer with software receives and displays the signal to a recipient.

### BACKGROUND OF THE INVENTION

1. Field of the Invention

This invention relates to a therapeutic safety device, and more specifically, to a hand grip device that easily fits within one's hand. More particularly, this invention relates generally to a device and method of providing a treatment and therapy by providing a grip device in one's hand by the user to provide a therapeutic effect and work out various muscles in one's hands as well as providing a safety device.

2. Related Art

The invention is valuable in the field of therapeutic treatment. Currently there are a various therapeutic devices for one's hand. Such devices as they relate to the instant invention include grip squeezers or safety devices for use in care giver settings.

### SUMMARY OF THE INVENTION

It is another object to improve the field of hand and arm therapy.

Still another object is to improve grip stability.

It is a further object to provide a device for increasing grip and arm strength.

It is another object to provide a safety device for use in care giver settings.

Another object is to provide a therapeutic device for arthritic patients.

Accordingly, in one embodiment, the invention is directed to a therapeutic grip device for use in one's hand. One aspect of the device includes a generally tube-like member with a lengthwise opening and having about hand-width to permit grasping thereabout with one's hand and wherein the tube-like member includes a spring aspect which is preformed to an open relaxed position and is compressible to a closed position to exert a force on one's hand muscles and wherein the device includes a thermal element which is equipped for generating a non-ambient temperature.

Preferably, the thermal element can be equipped with an on-demand component to generate a predetermined temperature when turned on. The thermal element can include a resistive element and power source or optionally a material which absorbs energy, such as from a microwave, and gives off heat over a period of time.

Another aspect of the device is directed to a safety grip device for use in one's hand. One aspect of the device includes a generally tube-like member with a lengthwise opening and having about hand-width to permit grasping thereabout with one's hand and wherein the tube-like member includes a spring aspect which is preformed to an open relaxed position and is compressible to a closed position to exert a force on one's hand muscles and includes a sensor and transmitter circuitry which when the safety grip device is compressed sends a signal indicative that compression is taking place. The invention provides for a computer based device to include operating software and hardware and compression alert software operably associated therewith, the computer based device being operably associated with the safety grip device to receive the signal from the safety grip device. The compression alert software is equipped for displaying the signal. Further, the compression alert software is equipped for relaying the compression signal to a recipient via various electronic methods, such as e-mail or text message.

The therapeutic grip device can also include a fastener to aid in retention in one's hand. The fastener can include a hook and loop type fastener, for example.

The cross-sectional configuration can include cylindrical or irregular ring, rectangular, square, multisided polygon including sides having equal or unequal lengths.

A method of providing therapy to a patient, which includes the steps of:

(a) holding in a hand of the patient a therapeutic grip device, wherein the therapeutic grip device includes generally tube-like member with a lengthwise opening and having about hand-width to permit grasping thereabout with one's hand and wherein the tube-like member includes a spring aspect which is preformed to an open relaxed position and is compressible to a closed position to exert a force on one's hand muscles and wherein the device includes a thermal element which is equipped for generating a non-ambient temperature; and

(b) compressing the therapeutic grip device by one's hand to works one's hand and arm muscles while the device gives off non-ambient temperature.

Another method of the invention is directed to a method of providing safety to a patient, which includes the steps of:

(a) placing in a hand of the patient a grip device, wherein the grip device includes generally tube-like member with a lengthwise opening and having about hand-width to permit grasping thereabout with one's hand and wherein the tube-like member includes a spring aspect which is preformed to an open relaxed position and is compressible to a closed position to exert a force on one's hand muscles and includes a sensor and transmitter circuitry which when the grip safety device is compressed sends a signal indicative that compression is taking place; and

(b) compressing the grip device by one's hand to cause the signal to be transmitted.

The method further includes the step of providing a computer based device which includes operating software and hardware and compression alert software operably associated therewith, the computer based device being operably associated with the safety grip device to receive the signal from the safety grip device. Another step provides for the compression alert software to display the signal. Still another step provides for the compression alert software to relaying the compression signal to a recipient, such as via various electronic methods, e.g. e-mail or text message.

Further aspects of the invention are evident from the Detailed Description of the Preferred Embodiments.

### BRIEF DESCRIPTION OF THE DRAWINGS

FIG. 1 is a perspective view of an embodiment of a grip device of the present invention an open uncompressed state;

FIG. 2 is a sectional view of FIG. 1 taken across line 2-2.

FIG. 3 is yet another perspective view of the device.

### DETAILED DESCRIPTION OF PREFERRED EMBODIMENTS

The invention is described in detail as preferred embodiments with the understanding that the present disclosure is to be considered as an exemplification of the principles of the invention and is not intended to limit the broad aspect of the invention to the embodiment illustrated. The grip device 10 of the present invention solves the problems associated with the prior art by providing the grip device 10 which therapy of the hands and serves as a safety device as will become apparent hereinafter. In one aspect of the invention, the grip device 10 is used in connection with providing therapy to one's hands. In another aspect, the grip device 10 is used to sense compression of the device and transmit a signal indicating compression of the device.

The grip device 10 includes outer covering 12 which includes jaw members 14 which are shown here as opposing generally arc shaped members separated by a lengthwise opening 18.

Outer covering 12 and can be formed from a natural or synthetic rubber material and can preferably include an antimicrobial material. The grip device 10 can preferably be of a length L of about a hand-width, e.g., about 4 inches, to permit grasping thereabout.

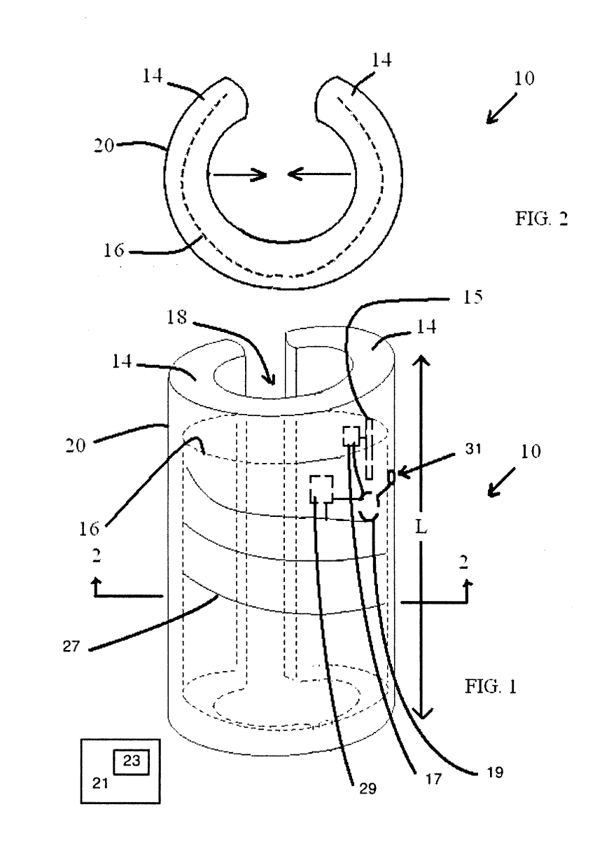

The jaw members 14 can include a spring element 16 which is preformed to an open position as seen in FIG. 1-5. The jaw members 14 and spring element 16 are compressible to a closed position as seen in FIG. 6. A sensor 15, such as pressure sensor (pressure transducer) or force sensor (a transducer that converts an input mechanical force into an electrical output signal), can be operably connected to the grip device 10. Here, the sensor 15 is operably disposed in connection with the spring element 16, although it is contemplated that it can be separately disposed in the grip device 10 when the spring element 16 is not a separate component of the grip device 10, but as an integral feature by virtue of the material of the grip device 10.

Additionally, the grip device 10 can preferably include transmitter circuitry 17 which is operably connected to said sensor 15 and both of which are operably connected to a power source, which can be a battery 19 internally stored within the grip device 10 or optionally can be an AC power supply configured with suitable power adapters to supply power to the sensor 15 and transmitting circuitry 17. When the safety grip device 10 is compressed, the sensor 15 and transmitting circuitry 17 enables sending a signal indicative that compression is taking place.

The invention provides for a computer based device 21 which includes operating software and hardware (processor, memory, power, etc.), and compression alert software 23 operably associated therewith, the computer based device 21 can be operably associated with the grip device 10 to receive the signal from the grip device 10. The compression alert software 23 is equipped for displaying the signal on a screen, for example. Further, the compression alert software 23 is equipped for relaying the compression signal to a recipient via various electronic methods, such as e-mail or text message.

The grip device can also include a fastener 25 to aid in retention in one's hand. The fastener 23 can include a hook and loop type fastener, for example.

A further aspect of the grip device 10 provides for a resistive element 27, such as a conductive wire, which when supplied with power, results in heat generation. Control circuitry 29 is operably connected to the resistive element 27 can be provided to selectively adjust temperature as desired. The thermal resistive element 27 via control circuitry 29 is equipped with an on-demand component to generate a predetermined temperature when turned on via switch 31.

When compressed in a direction of arrows seen in FIG. 2, the grip device 10 exerts a force on one's hand and forearm muscles. In one case, the spring 16 which can include a polymer spring, such as a polypropylene and nylon composition or spring metal formed therein. The spring 16 which can be inserted into a mold in a manner to be held centrally such that outer covering 12 is formed by injecting uncured material about the spring 16 wherein it cures about the spring 16.

Optionally, rubber or other polymer can also be formed in a manner as a composite laminate to delivery memory to perform the invention as a single component. The jaw members 14 can be formed with arcuate surface or other desirable surface configuration which lends itself to use for carrying out the purposes of the invention.

In this embodiment, the device 10 is of about a hand width, and can be slightly less to accommodate children to adults, such as several inches up to about four inches for a single hand usage. It is recognized that the thickness of the device 10 can be varied to accommodate user preference. Additionally, the amount of resistance can be varied to accomplish a particular use or therapy.

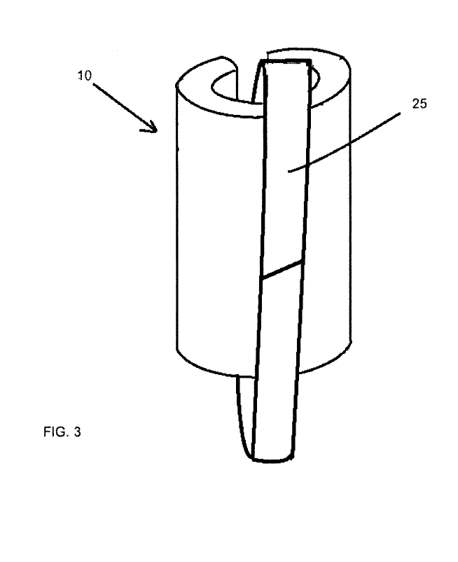

The outer covering 20 can serve as a pad having an outward gripping surface and an opposing inner surface. The outer covering 20 can be textured (as seen in FIG. 3) for functional an aesthetic purposes. The outer covering 20 and spring 16 can be formed from in varying amount of compressibility and spring force to accommodate user preferences. For example, natural or synthetic rubber or polyfoam can be used and can optionally include an antimicrobial material.

It is contemplated that the orientation of the spring 16 relative to the covering 20 can be varied. An exemplary version contemplates a non-metal outer covering which in the case of a laminate could comprise the spring material internally. The outer gripping surface of covering 20 can be of a tread design to enhance hand grip.

However, the element 16 can be recessed within the outer covering 20. Further, the spring 16 provides for not only strength but also flexibility in the overall product and enables a various materials to be employed to carry out this aspect such as nylon, polypropylene or polymer blend or spring metal (which could also serve as a part of resistive component), for example, within a natural or synthetic rubber outer cover.

By employing the instant invention, the invention provides for therapeutic treatment for muscle training. Further, the invention provides a safety or alert device in situations wherein a person is in a state or condition to be monitored, such as a comatose or sedated state, wherein an attendant would desire to know when such person is becoming alert typically by one's hands movement being sufficient to compress the device 10. In this way the person would not need to be restrained in conventional manners, rather the device 10 can be placed in each hand and maintained via wrapping a hook and loop strap 25 around the device 10 and about the back of the person's hand so that the device 10 is maintained in place. The strap 25 can also include a hydrogel material for ease of use on skin of the hand.

While specific embodiments have been illustrated and described, numerous modifications come to mind without markedly departing from the spirit of the invention. The invention covers a tube-like member having an opening running axially lengthwise wherein the tube-like member can be formed with a spring material and take on geometric shapes which are suitable for practicing the invention. The non-ambient temperature can include heat and cold. In this regard, it is contemplated the thermal component of the invention can include the material of the outer covering 20 which can include a thermal absorbing matter to retain heat or cold (such as a thermo-gel such as Futuro® from 3M) in the alternative to a rubber covering which includes the resistance element 27 for heating the device 10. Thus there is provided a therapeutic device which is highly suitable for those suffering from poor circulation or arthritis or serves as a patient safety alert for care givers. It is contemplated the device 10 can be made in a disposable or reusable manner. By keeping sensitive components contained within the covering 20, the device 10 lends itself to sterilization techniques such as with ethylene oxide.

The scope of protection is thus only intended to be limited by the scope of the accompanying claims. Thus, the breadth and scope of the present invention should not be limited by any of the above described exemplary embodiments, but should be defined only in accordance with the following claims and their equivalents.

1. A grip device for use in one's hand, which includes:
a compressible member of a size to fit within one's hand and capable of being squeezed by the hand; and
a thermal element operably connected to said compressible member which is equipped for generating a non-ambient temperature for a sustained period during such period said device remains compressible.

2. The grip device of claim 1, wherein said compressible member is a generally tube-like member with a lengthwise opening and having about hand-width to permit grasping thereabout with one's hand and wherein the tube-like member includes a spring aspect which is preformed to an open relaxed position and is compressible to a closed position to exert a force on one's hand muscles.

3. The grip device of claim 1, wherein said thermal element is equipped with an on-demand component to generate a predetermined temperature when turned on.

4. The grip device of claim 1, wherein said thermal element includes a resistive element and power source.

5. The grip device of claim 1, wherein said thermal element includes a material which absorbs energy and releases energy as said non-ambient temperature.

6. The grip device of claim 1, which includes a sensor and transmitter circuitry which when said grip device is compressed sends a signal indicative that compression is taking place.

7. The grip device of claim 6, which includes a computer based device having operating software and hardware and compression alert software operably associated therewith, said computer based device being operably associated with said safety grip device to receive said signal from said grip device.

8. A grip device for use in one's hand, which includes:
a member of a size to fit within one's hand and capable of being squeezed by the hand; and
a sensor and transmitter circuitry operably connected to said member which when said grip device is squeezed sends a signal indicative that squeezing is taking place.

9. The grip device of claim 8, wherein said member is a compressible generally tube-like member with a lengthwise opening and having about hand-width to permit grasping thereabout with one's hand and wherein the tube-like member includes a spring aspect which is preformed to an open relaxed position and is compressible to a closed position to exert a force on one's hand muscles.

10. The grip device of claim 8, which includes a computer based device having operating software and hardware and squeezing alert software operably associated therewith, said computer based device being operably associated with said grip device to receive said signal from said grip device.

11. The grip device of claim 10, wherein said squeezing alert software is equipped for displaying the signal.

12. The grip device of claim 11, wherein said alert software is equipped for relaying said squeezing signal to a recipient via one of an e-mail and text message.

13. The grip device of claim 8, which includes a fastener to aid in retaining said grip device to one's hand.

14. The grip device of claim 13, wherein said fastener includes a hook and loop type fastener.

15. The grip device of claim 13, wherein said fastener includes a hydrogel material.

16. A method of providing therapy to a patient, which includes the steps of:
(a) holding in a hand of the patient a therapeutic grip device, wherein the therapeutic grip device includes a member of a size to fit within one's hand and capable of being squeezed by the hand and a thermal element which is equipped for generating a non-ambient temperature; and
(b) squeezing the therapeutic grip device by one's hand to works one's hand and arm muscles while the device gives off non-ambient temperature.

17. A method of providing safety to a patient, which includes the steps of:
(a) placing in a hand of the patient a grip device, wherein the grip device includes a member of a size to fit within one's hand and capable of being squeezed by the hand and a sensor and transmitter circuitry which when said grip device is squeezed sends a signal indicative that squeezing is taking place; and
(b) squeezing said grip device by one's hand to cause the signal to be transmitted.

18. The method of claim 17, which further includes the step of providing a computer based device having operating software and hardware and squeezing alert software operably associated therewith, said computer based device being operably associated with the grip device to receive said signal from said grip device.

19. The method of claim 18, wherein said squeezing alert software displays said signal.

20. The method of claim 18, wherein said squeezing alert software relays said squeezing signal to a recipient via one of e-mail and text message.

21. A method of providing safety to a patient, which includes the steps of:
(a) placing in a hand of the patient a grip device, wherein the grip device includes a member of a size to fit within one's hand which when so retained precludes such hand from grasping another object until said grip device is removed squeezed; and
(b) removably fastening said grip device to the hand.
